(19) United States
(12) Patent Application Publication
(10) Pub. No.: US 2024/0398437 A1
(43) Pub. Date: Dec. 5, 2024

(54) METHOD AND DEVICE FOR DRIVING ULTRASONIC SURGICAL INSTRUMENT, AND ULTRASONIC SURGICAL SYSTEM

(71) Applicant: Reach Surgical, Inc., Tianjin (CN)

(72) Inventors: Fengtang Li, Tianjin (CN); Qiang Yang, Tianjin (CN); Xiaoyun Fan, Tianjin (CN); Sandong Li, Tianjin (CN)

(73) Assignee: Reach Surgical, Inc., Tianjin (CN)

(21) Appl. No.: 18/799,068

(22) Filed: Aug. 9, 2024

Related U.S. Application Data
(63) Continuation of application No. PCT/CN2023/075246, filed on Feb. 9, 2023.

(30) Foreign Application Priority Data
Feb. 11, 2022 (CN) 202210130249.9

(51) Int. Cl. A61B 17/32; A61B 17/00

(52) U.S. Cl. CPC A61B 17/320068; A61B 2017/00026; A61B 2017/00141; A61B 2017/320074; A61B 2017/320078

(57) ABSTRACT

A method comprises steps of in response to an initiating signal, providing a drive signal with a first amplitude range for a first period to drive an transducer of the ultrasonic surgical instrument; subsequent to the first period, providing a first transition signal with a decreasing amplitude for a first predetermined interval; subsequent to the first predetermined interval, providing a drive signal with a second amplitude range for a second period, wherein an upper limit of the second amplitude range is lower than a lower limit of the first amplitude range; subsequent to the second period, providing a second transition signal with an increasing amplitude for a second predetermined interval; and subsequent to the second predetermined interval, providing a drive signal with a third amplitude range for a third period, wherein a lower limit of the third amplitude range is higher than the upper limit of the second amplitude range.

### CROSS-REFERENCE TO RELATED PATENT APPLICATIONS

This application is a continuation of International Application No. PCT/CN2023/075246, filed Feb. 9, 2023, which claims the benefit of and priority to Chinese Patent Application No. 202210130249.9, filed Feb. 11, 2022, the entire disclosures of both of which are incorporated herein in their entireties, including their specifications, drawings, claims, and abstracts.

### BACKGROUND

The present application relates to the field of surgical instruments, and more particularly, to a method and a device for driving an ultrasonic surgical instrument, and an ultrasonic surgical system.

Ultrasonic surgical instruments, also known as ultrasonic soft tissue cutting and/or coagulation system, mainly uses ultrasonic energy to promote soft tissue cutting and coagulation to stop bleeding, and have less thermal damage, which is suitable for cutting human soft tissues except bone tissues and fallopian tubes. The ultrasonic surgical instrument and generator are key members of the ultrasonic surgical system, wherein the ultrasonic surgical instrument comprises a transducer and an effector. The effector comprises a blade and a jaw assembly, and the effector is connected to the transducer through transmission media. When working, the generator outputs driving signals to the transducer according to specific frequency and amplitude, and the transducer converts the driving signals into mechanical vibration. The mechanical vibration is transmitted and amplified by the transmission media and then transmitted to the blade in the effector, so that the blade vibrates at ultrasonic frequency. Operators can operate the tissue part by operating the blade and the jaw assembly cooperatively to form appropriate pressure, or directly apply the blade to the tissue part for operation.

Due to the advantages in hemostasis and thermal injury, the ultrasonic surgical instrument is often used to seal vessel. The process of sealing vessel comprises three stages: separating muscle tissue layer of vessel, sealing and coagulating vessel, and cutting vessel. In different stages, the amplitude and frequency of the driving signal output by the generator are also different, thus changing the vibration amplitude and frequency of the blade. Combined with a hand-held force exerted by the operator, an acting force between the blade and the jaw assembly is changed, which meets the requirements of applying force to vessels in different stages. According to the three stages of sealing vessel, the driving signal output by the generator in the existing solution is a step signal. The amplitude of the signal instantly jumps from a high point to a low point when entering the second stage from the first stage, and the amplitude of the signal instantly jumps from a low point to a high point when entering the third stage from the second stage. This leads to a sudden change of the ultrasonic vibration energy of the blade, which will not only affect the stability of force applied by the operator and the manipulation accuracy in the process of sealing vessel, but also affect the tearing force acting on the tissue part, and may also cause the blade to break.

### SUMMARY

It is provided in the present application a method and device for driving an ultrasonic surgical instrument, and an ultrasonic surgical system. The method comprises steps of: in response to an initiating signal, providing a drive signal with a first amplitude range to the ultrasonic surgical instrument for a first period to drive a transducer of the ultrasonic surgical instrument; subsequent to the first period, providing a first transition signal with a decreasing amplitude for a first predetermined interval to drive the transducer; subsequent to the first predetermined interval, providing a drive signal with a second amplitude range for a second period to drive the transducer, wherein an upper limit of the second amplitude range is lower than a lower limit of the first amplitude range; subsequent to the second period, providing a second transition signal with an increasing amplitude for a second predetermined interval to drive the transducer; and subsequent to the second predetermined interval, providing a drive signal with a third amplitude range for a third period to drive the transducer, wherein a lower limit of the third amplitude range is higher than the upper limit of the second amplitude range; wherein, at least one of the first predetermined interval and the second predetermined interval is above zero (i.e. greater than zero).

In addition, it is provided in the present application a generator for driving an ultrasonic surgical instrument. The generator comprises a processor chip, the processor chip is pre-loaded with program information, and when the generator responds to an initiating signal, the processor chip executes the program information to perform the method for driving the ultrasonic surgical instrument according to the solution above, so that the generator outputs a driving signal to the ultrasonic surgical instrument.

Furthermore, it is provided in the present application an ultrasonic surgical system, comprising an ultrasonic surgical instrument and the generator according to the solution above, wherein: the ultrasonic surgical instrument comprises a transducer and an operating assembly; the transducer receives the driving signal output by the generator and converts the driving signal into an ultrasonic vibration signal; and the operating assembly is provided with a waveguide and an end effector assembly arranged at a distal end of the waveguide and used for operating a tissue; and the ultrasonic vibration signal is transmitted to the end effector assembly from the waveguide, and ultrasonic energy generated by the end effector assembly acts on the operated tissue.

### BRIEF DESCRIPTION OF THE DRAWINGS

While the specification concludes with claims particularly pointing out and distinctly claiming the disclosure, it is believed that the disclosure will be better understood from the detailed description of certain embodiments thereof when read in conjunction with the accompanying drawings. Unless the context indicates otherwise, like numerals are used in the drawings to identify similar elements in the drawings. In addition, some of the figures may have been simplified by the omission of certain elements in order to more clearly show other elements. Such omissions are not necessarily indicative of the presence or absence of particular elements in any of the exemplary embodiments, except as may be explicitly stated in the corresponding detailed description.

FIG. 1 is a schematic diagram of an integral structure of an ultrasonic surgical instrument according to one embodiment of the present application;

FIG. 2 is a schematic structural diagram of a transmission assembly according to one embodiment of the present application;

FIG. 3 is a schematic diagram of an ultrasonic energy generation and transmission process of the ultrasonic surgical instrument according to one embodiment of the present application;

FIG. 4 is a flow chart of a method for driving the ultrasonic surgical instrument according to one embodiment of the present application;

FIG. 5a to FIG. 5e are schematic diagrams of waveform of a driving signal according to embodiments of the present application;

FIG. 6 is a view showing the curve of changes in vessel wall thickness in a process of sealing vessel according to the embodiments of the present application;

FIG. 7 is a schematic diagram of waveform of the driving signal according to the embodiments of the present application;

FIG. 8 is a flow chart of adjusting the signal in the method for driving the ultrasonic surgical instrument according to one embodiment of the present application;

FIG. 9 is a schematic diagram of waveform of a driving signal according to another embodiment of the present application;

FIG. 10a and FIG. 10b are structural block diagrams of the ultrasonic surgical system according to the embodiments of the present disclosure;

FIG. 11 is an exploded view of an end effector assembly in a partially closed state according to one embodiment of the present application;

FIG. 12 is an exploded view of the end effector assembly shown in FIG. 11 in a partially opened state;

FIG. 13 is a schematic diagram of the end effector assembly in the partially closed state according to one embodiment of the present application;

FIG. 14a and FIG. 14b are comparison diagrams of deformations of the ultrasonic surgical instruments of the prior art and the present application when the end effector assembly clamps the vessel; and

FIG. 15a to FIG. 15c are comparison diagrams of experimental results of sealing vessel between the present application and the prior art.

### DETAILED DESCRIPTION

The following detailed description describes examples of embodiments of the disclosure solely for the purpose of enabling one of ordinary skill in the relevant art to make and use the invention. As such, the detailed description and illustration of these embodiments are purely illustrative in nature and are in no way intended to limit the scope of the disclosure, or its protection, in any manner. It should also be understood that the drawings are not to scale and in certain instances details have been omitted, which are not necessary for an understanding of the present disclosure.

In the description of the present application, it should be noted that, the orientation or positional relationships indicated by the terms “center”, “above”, “below”, “left”, “right”, “vertical”, “horizontal”, “inside”, “outside” and the like are orientation or positional relationships based on the accompanying drawings, which are only for convenience and simplification of the description of the present application, but are not intended to indicate or imply that the indicated device or element must have a specific orientation, be constructed and operated in a specific orientation, and thus, cannot be understood as a limitation to the present application. Moreover, the terms “first”, “second” and “third” are used for descriptive purposes only and cannot be understood as indicating or implying relative importance.

In the description of the present application, it should be noted that unless otherwise specified and limited, the terms “mounted”, “in connection with”, “connected” and the like should be understood broadly, for example, the connection may be fixed connection, and may also be detachable connection or integral connection; may be direct connection, may also be indirect connection through an intermediate medium, and may also be internal communication of two elements. The specific meanings of the above terms in the present application can be understood by those of ordinary skills in the art according to specific conditions. In addition, the technical features involved in different embodiments of the present application described below can be combined with each other as long as they do not constitute conflicts with each other.

In various embodiments of the present application, “distal end/side” refers to an end/side of a surgical instrument that is far away from an operator when the surgical instrument is operated, and “proximal end/side” refers to an end/side that is close to the operator when the surgical instrument is operated.

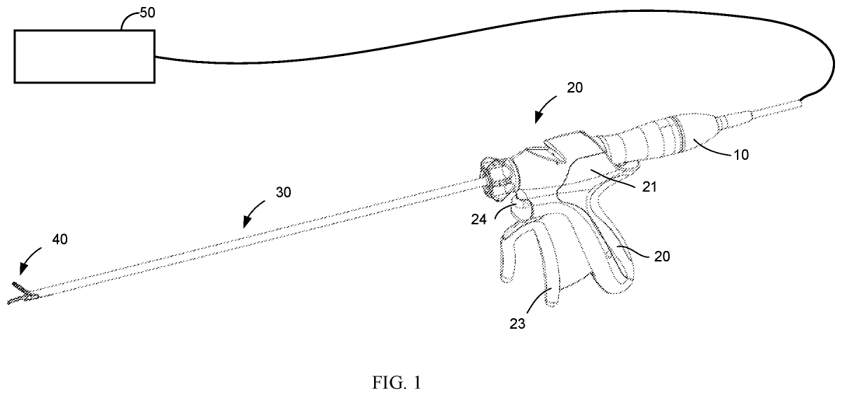

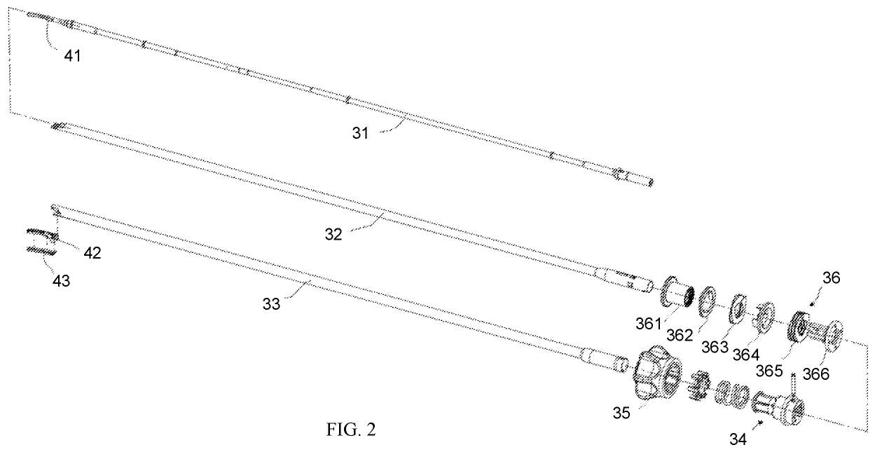

The following embodiments of the present application generally relate to an ultrasonic surgical system, which may be used to transect tissue, coagulate tissue and/or clamp tissue during surgeries. FIG. 1 to FIG. 3 show schematic structural diagrams of a specific embodiment of the ultrasonic surgical system of the present application. The ultrasonic surgical system comprises an ultrasonic surgical instrument. The ultrasonic surgical instrument comprises an operating assembly and a transducer 10. The operating assembly comprises a handle assembly 20, a transmission assembly 30, and an end effector assembly 40 which are sequentially arranged from a proximal end to a distal end. By inserting the transducer 10 into the handle assembly 20, the proximal end of the operating assembly is connected to a distal end of the transducer 10 and assembled together with the distal end. The ultrasonic surgical system further comprises a generator 50 configured for providing ultrasonic energy. The transducer 10 is operatively connected to the generator 50 through a cable, which converts electrical energy from the generator 50 into ultrasonic vibration that is further transmitted to the end effector assembly 20. The handle assembly 20 is designed for an operator to operate the surgical instrument. For example, the handle assembly 20 is adapted to actuate the end effector assembly 40 through the transmission assembly 30, so as to conduct cutting and/or coagulation operations. The handle assembly 20 may be grasped by the operator in various manners. In a specific embodiment, the operating assembly of the ultrasonic surgical instrument is in a trigger-like arrangement to control the end effector assembly 40 to open and close.

The handle assembly 20 comprises a main housing 21 and a handgrip 22 downwardly extending from the main housing 21. The handle assembly 20, especially the handgrip 22 thereof, is adapted for being held by a medical practitioner, so that during use in order toa facilitate grasping and manipulation of the instrument, while isolating the operator from the ultrasonic vibrations. A trigger 23 is supported on the handle assembly 20 for pivotal movement towards and away from the handgrip 22 to cause pivotal movement of the clamp arm assembly 42 located at the distal end of the transmission assembly 30. The handle assembly 20 is provided with a hand switch 24, and is used to be pressed toward the handgrip 22 to control operation of the instrument, for example, allowing the ultrasonic generator to output, so as to cause the ultrasonic blade 41 to vibrate. The end effector assembly 40 clamps the tissue (i.e., applies pressure to the tissue) during closing of the end effector assembly 40, and at the same time, under the action of the ultrasonic energy, performs tissue cutting and coagulation. The force acting on the tissue in manner of ultrasonic energy may be defined as loading force. A proximal end of the main housing 21 is open so as to receive the ultrasonic transducer 10 to be inserted therein.

The end effector assembly 40 comprises a blade 41 and a jaw assembly 42 (also called clamp arm) that may be operatively actuated for pivotal movement towards or away from the ultrasonic blade 41, so as to close or open the end effector assembly 40. The jaw assembly 42 may be actuated to move between an open position and a closed position. In the open position, at least a part of the jaw assembly 42 is actuated to be spaced from the blade 41. In the closed position, the jaw assembly 42 urges against the tissue clamped between the jaw assembly 42 and the blade 41.

The transmission assembly 30 may be configured as an elongated shaft extending distally away from the handle assembly 20 of the instrument. The transmission assembly 30 comprises a waveguide 31 for transmitting the ultrasonic energy provided by the transducer 10 to the blade 41, an inner tube 32 sleeved on the waveguide 31, and an outer tube 33 sleeved on the inner tube 32. The outer tube 33 is axially mounted relative to the handle assembly 20, and the jaw assembly 42 is pivotally connected to the outer tube 33. The proximal portion of the inner tube 32 is coupled with an actuation mechanism of the handle assembly 20, and the distal portion is couple to the jaw assembly 42. The inner tube 32 is actuated through the actuation mechanism so as to be reciprocated axially relative to the outer tube 33, and further actuate the jaw assembly 42 to pivot about the outer tube 33. The jaw assembly 42 comprises a clamp pad 43. The jaw assembly 42 is connected to distal ends of the outer tube 33 and the inner tube 32 together with the clamp pad 43. The clamp pad 43 is supported on the jaw assembly 42 for matching with the blade 41, and pivotal movement of the jaw assembly 42 positions the clamp pad 43 substantially parallel to and in contact with the blade 41, thereby defining a tissue treatment area. With this structure, the tissue is clamped between the clamp pad 43 and the blade 41.

Proximal ends of the waveguide 31, the outer tube 33 and the inner tube 32 are connected to each other through a bayonet connector assembly located in the main housing 21, so that the waveguide 31, the outer tube 33 and the inner tube 32, as a whole, may rotate together with the ultrasonic transducer 10 relative to the handle assembly 20 by means of a knob 35. The waveguide 31 extends into the main housing 21 of the handle assembly 20 through the knob 35. During usage, the outer tube 33 and the waveguide 31 can be rotated through rotation of the knob 35, so that the end effector assembly 40 and the jaw assembly 42 connected thereto are adjusted to a required direction. During usage, the rotation of the knob 35 relative to the handle assembly 20 causes the outer tube 33, the waveguide 31 and the ultrasonic transducer 10 operatively connected thereto to rotate relative to the handle assembly 20.

A reciprocating motion of the inner tube 32 drives the jaw assembly 42 to open or close. A force limiting mechanism 36 is operatively connected to the inner tube 32, and comprises a tube collar cap 361. A distal washer 362, a distal wave spring 363, a proximal washer 364 and a proximal wave spring 365 are fastened to a collar 366 by the tube collar cap 361. The collar 366 comprises an axially extending lug, and the lug is engaged with an appropriate opening in a proximal portion of the tubular inner tube 32. The inner tube 32 receives an O-shaped ring 367 at a circumferential groove, and the O-shaped ring is used for being engaged with an inner surface of the outer tube 33.

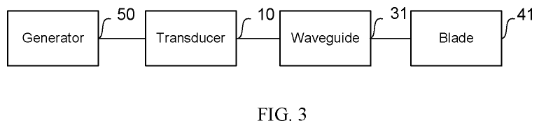

Based on the above description of the structure of the ultrasonic surgical system, a schematic diagram of an ultrasonic energy transmission process as shown in FIG. 3 can be obtained. The generator 50 outputs a driving signal, which is an signal with a specific current and frequency. The transducer 10 converts the driving signal into an ultrasonic vibration signal, and the ultrasonic vibration signal (ultrasonic energy) can be transmitted to the waveguide 31 by an operating hand switch 24. The waveguide 31 is suitable for transmitting the ultrasonic energy from the transducer 10 to the blade 41 located at the distal end of the waveguide 31, wherein the waveguide 31 may be flexible, semi-flexible or rigid. As known to those skilled in the art, an amplitude and/or frequency of a vibration wave propagating along a length direction of the waveguide 31 can be adjusted by changing a diameter of the waveguide 31 or other corresponding characteristics. For example, reducing the diameter, especially the diameter of a vibration node of the waveguide or the diameter near a vibration node of the waveguide, can increase an amplitude of the mechanical vibration transmitted from the waveguide 31 to the blade 41. Other features may also be provided on the waveguide 31 to control the gain (positive or negative) of longitudinal vibration transmitted along the waveguide, and to adjust the vibration of the waveguide 31 to an ideal resonance frequency for the system. In this way, the waveguide 31 may have different cross-sectional sizes, or comprise substantially uniform cross-sections, or be tapered at a plurality of positions along the waveguide 31 to provide two or more sections with different cross-sections, or even be tapered along an entire length of the waveguide.

In various embodiments, the waveguide 31 may be made from a variety of materials, particularly various medically and surgically acceptable metals such as titanium, titanium alloy (for example, Ti6A14V), aluminum, aluminum alloy or stainless steel. In some embodiments, such as the embodiments shown in the drawings, the blade 41 and the waveguide 31 are formed as a single unit, for example, such as fabricated from a single metal rod that has been milled so as to provide the desired features. Alternately, the waveguide 31 and the ultrasonic blade 41 may comprise two or more separable components of the same of differing compositions, with the components coupled to one another by, for example, adhesive, welding, a threaded stud, and/or other suitable ways known to those skilled in the art. For example, the ultrasonic blade 41 may be connected to the waveguide by a threaded connection, a welded joint, or other coupling mechanisms.

By way of example, generator 50 and transducer 10 in the depicted embodiment are configured to generate a standing vibrational wave having a frequency of about 55 kHz. However, various other ultrasonic frequencies may be employed, such as between about 20 and about 120 kHz.

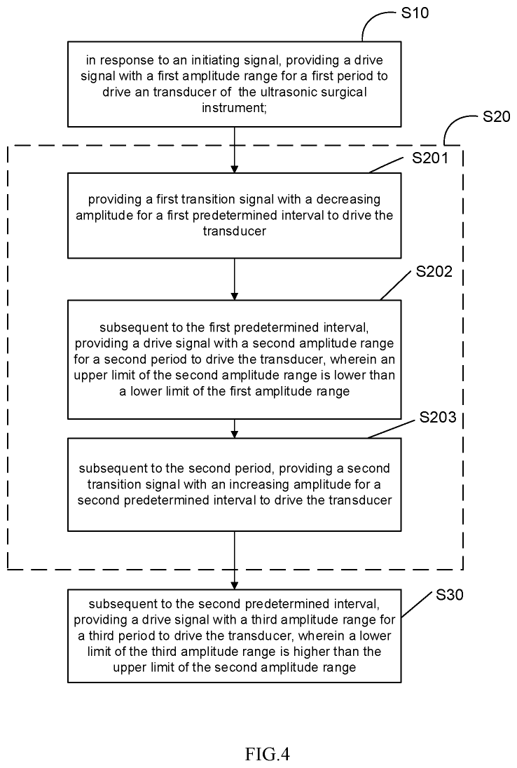

It is provided in various embodiments of the present application a method for driving an ultrasonic surgical instrument. As shown in FIG. 4, the method comprises the following steps.

At S10, this step is for separating the muscle layer of the vessel, which comprises: in response to an initiating signal, providing a drive signal with a first amplitude range for a first period to drive the transducer of the ultrasonic surgical instrument. In this step, the transducer is actuated by the drive signal with the first amplitude range, so as to cause the blade 41 to vibrate with a relatively high ultrasonic vibration energy, for rapid drying the wall of the vessel.

At S20, this step is for sealing and/or coagulation of the vessel, which comprises: S201: subsequent to the first period, providing a first transition signal with a decreasing amplitude for a first predetermined interval to drive the transducer; S202: subsequent to the first predetermined interval, providing a drive signal with a second amplitude range for a second period to drive the transducer, wherein an upper limit of the second amplitude range is lower than a lower limit of the first amplitude range; and S203: subsequent to the second period, providing a second transition signal with an increasing amplitude for a second predetermined interval to actuate the transducer.

The value(s) of the first predetermined interval and/or the second predetermined interval is(are) above zero (i.e. greater than zero). For example, if the first predetermined interval is determined to be zero, the termination of the first period is taken as the beginning of the second period; alternatively, if the second predetermined interval is determined to be zero, the termination of the second period is taken as the beginning of the third period. During this step, the amplitude of the drive signal for driving the transducer of the ultrasonic surgical instrument is lower than that of the drive signal in step S10, so as to cause the blade 41 to vibrate with a lower ultrasonic vibration energy, for sealing or coagulation of the vessel.

At S30, this step is for cutting of the vessel, which comprises: subsequent to the second predetermined interval, providing a drive signal with a third amplitude range for a third period to actuate the transducer, wherein a lower limit of the third amplitude range is higher than the upper limit of the second amplitude range. During this step, the amplitude of the drive signal for driving the transducer is higher than that of the driving signal in step S20, so as to cause the blade 41 to vibrate with a higher ultrasonic vibration energy, for cutting the vessel.

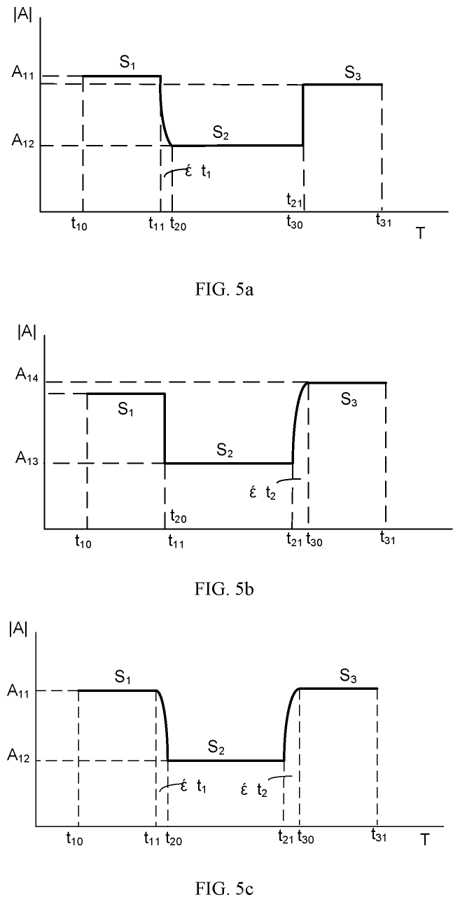

FIG. 5a to FIG. 5e are schematic diagrams showing various signal waveforms of the signals used for driving the ultrasonic surgical instrument in the above method. In general, the driving signals all comprise a first signal S1 for a first period, a second signal S2 for a second period and a third signal S3 for a third period. Under the driving of different signals, the operation of the end effector assembly 40 corresponds to different stages of vessel sealing.

More specifically, for the stage of separating the muscle layer of the vessel, the transducer is driven by the first signal S1 for the first period, and the end effector assembly 40 is actuated to perform separating the muscle layer of the vessel. During this stage, walls of the vessel is quickly dried. The drive signal for the first period is provided with a relative high amplitude, providing the end effector assembly 40 with a relative high energy, which facilitate separation of the muscle layer. As shown in the figures, t10 refers to the starting time of the first period, and t11 refers to the termination time of the first period.

The stage of sealing or coagulation the vessel starts from the termination time t11 of the first period to the starting time t30 of the third period. During this stage, the transducer is driven by the second signal S2, the transition signal between the second signal S2 and the first signal S1, and the transition signal between the second signal S2 and the third signal S3, so as to cause the end effector assembly 40 to vibrate for sealing or coagulation of the vessel. During this stage, the second signal S2 is provided with the second amplitude range, the upper limit of which is lower than the lower limit of the first amplitude range. The starting time of the second period is t20, and the termination time of the second period is t21. The drive signal for the second period is provided with a low amplitude, providing the end effector assembly 40 with a relative low energy, which facilitate sealing or coagulation of the vessel, preventing the carbonation of vessel wall caused by excessive energy of the end effector assembly 40.

For the stage of cutting the vessel, the transducer is driven by the third signal S3 for the third period, and the end effector assembly 40 is actuated to perform vessel cutting. During this stage, the drive signal is provided with the third amplitude range for the third period, the lower limit of which is higher than the upper limit of the second amplitude range. As shown in the figures, t30 refers to the starting time of the third period, and t31 refers to the termination time of the third period. The drive signal for this stage is provided with a relative high amplitude, providing the end effector assembly 40 with a relative high energy, which facilitate quick cutting of the vessel.

As shown in the figures, a drive signal with a decreasing amplitude is provided for a first predetermined interval Δt1 that is between the starting time t20 of the second period and the termination time t11 of the first period, and/or a drive signal with a increasing amplitude is provided for a second predetermined interval Δt2 between the starting time t30 of the third period and the termination time t21 of the second period. That is, the amplitude of the driving signal changes more smoothly, avoiding step changes of the drive signal. FIG. 5a shows a situation of setting a transition time when the first signal S1 is changing to the second signal S2. As shown in the figure, at the termination time t11 of the first period, the amplitude of the signal is A11; at the starting time t20 of the second period, the amplitude of the signal is A12. In the process of changing the amplitude of the signal from A11 to A12, the first predetermined interval Δt1 is defined as the transition time. FIG. 5b shows a situation of setting a transition time for the change phase from the second signal S2 to the third signal S3. As shown in the figure, at the termination time t21 of the second period, the amplitude of the signal is A13; at the starting time t30 of the third period, the amplitude of the signal is A14. In the process of changing the amplitude of the signal from A13 to A14, the second predetermined interval Δt2 is defined as the transition time. FIG. 5c to FIG. 5e show the situations where the transition time is set before the starting time t20 of the second period and after the termination time t21 of the second period. In the waveforms shown in the above figures, the amplitudes of the signals of the first signal S1 for the first period, the second signal S2 for the second period and the third signal S3 for the third period are all kept constant, and the amplitude of the signal of the third signal S3 may be different from that of the first signal S1 for the first period (as shown in FIG. 5a and FIG. 5b) or the same (as shown in FIG. 5c to FIG. 5e). However, in the practical application of this method, at each stage, the amplitude of the signal may fluctuate to some extent, that is, it is not stable at the amplitude A11 or A12, A13 or A14, which will be further explained in the following embodiments. When the amplitudes of the signals of the first signal S1 for the first period, the second signal S2 for the second period and the third signal S3 for the third period are all kept constant, and the amplitude of the signal of the third period signal S3 is the same as that of the first signal S1 for the first period, then A13=A12, and A14=A11. In this case, the signal waveforms shown in FIG. 5c to FIG. 5e can be obtained. In this embodiment, the above figures are mainly used to illustrate the existence of the first predetermined interval Δt1 and/or the second predetermined interval Δt2. An alternative solution is to predetermined intervals for the change between the two phases to realize the slow transition of the amplitude change of the signal.

According to the above solution of this embodiment, the first predetermined interval Δt1 is set between the first signal S1 and the second signal S2 for transition, or the second predetermined interval Δt2 is set between the second signal S2 and the third signal S3 for transition, or the transition time is set for changes in both phases at the same time, and the step change signals in the two phases are partially or completely adjusted to transition within a certain period of time. Therefore, it has a certain buffer when the ultrasonic energy transmitted to the blade 41 changing between high power and low power, which can effectively improve operating accuracy of the operator on the ultrasonic instrument, reduce the impact on sudden change of a tissue tearing force, and avoid the blade from breakage.

In the above solution, when the first predetermined interval Δt1 and the second predetermined interval Δt2 are provided, the amplitude of the signal changes linearly (as shown in FIG. 5d) or nonlinearly (as shown in FIG. 5a to FIG. 5c and FIG. 5e).

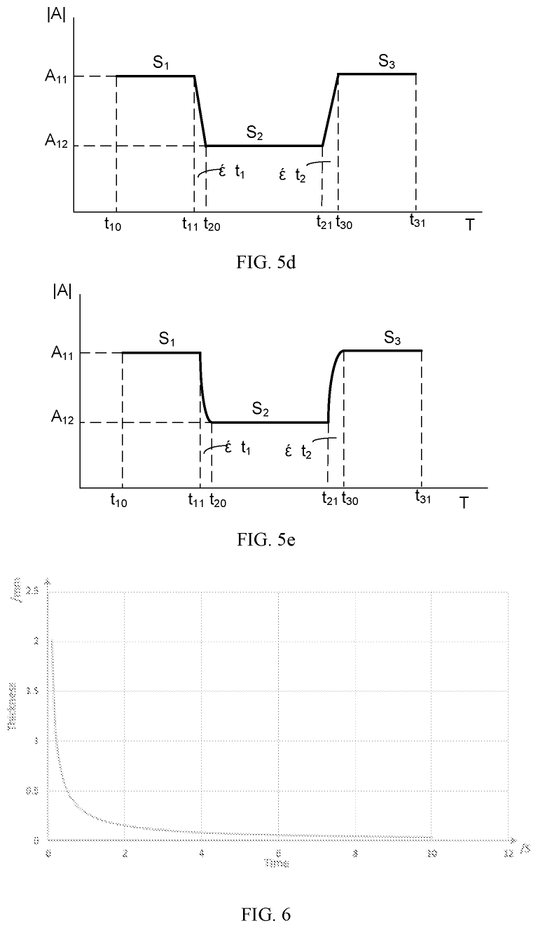

In order to shorten operating time for transecting large diameter vessels, as well as successfully sealing or coagulating the vessel, loading force shall be provided corresponding to the thickness of the vessel wall. For example, the amplitude of the transition signal between the second signal S2 and the first signal S1 as well as the amplitude of the transition signal between the second signal S2 and the third signal S3 change nonlinearly, so as to better match change of the thickness of the vessel wall and conduct the coagulation and cutting of the vessel. That is, in the case that the first predetermined interval Δt1 is greater than zero or above zero, the amplitude of the first transition signal provided to the ultrasonic surgical instrument over the first predetermined interval Δt1 decreases following a first nonlinear curve; in the case that the second predetermined interval Δt2 is greater than zero or above zero, the amplitude of the second transition signal provided to the ultrasonic surgical instrument over the second predetermined interval Δt2 increases following a second nonlinear curve, so as to make the changes of the wall thickness of the vessel follow the curve shown in FIG. 6 during sealing process.

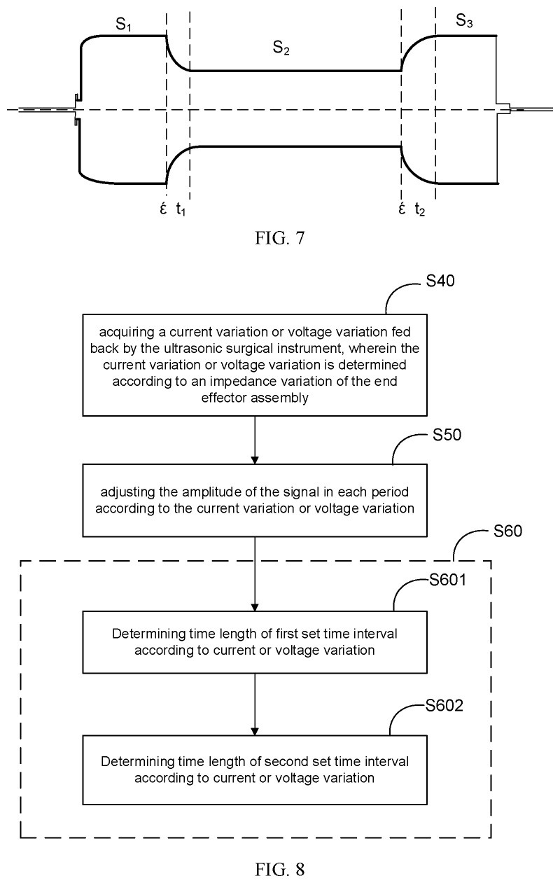

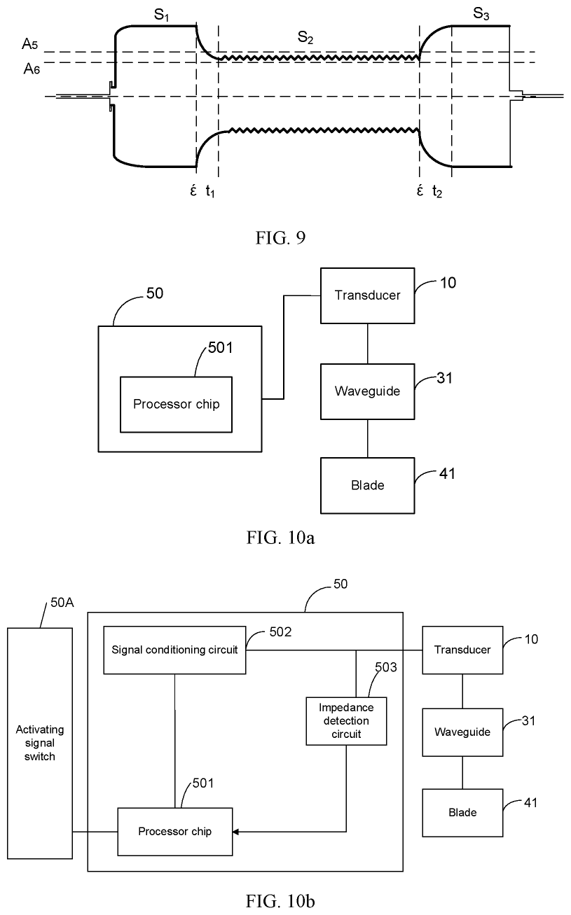

Specifically, the waveforms of the first transition signal and the second transition signal provided in this embodiment are shown in FIG. 7. In which, the expression of the first nonlinear curve is: A=(A11−A12)×sin Φ+A11, Φ∈(180°, 270°), wherein A is the amplitude of the first transition signal, A11 is the amplitude of the signal at the termination time of the first period, A12 is the amplitude of the signal at the starting time of the second period, the starting time Φ of the first predetermined interval Δt1 is 180°, and the termination time Φ of the first predetermined interval Δt1 is 270°. The second nonlinear curve is: C=(A14−A13)×sin Φ+A13, Φ∈(0°, 90°), wherein C is the amplitude of the second transition signal, A13 is the amplitude of the signal at the termination time of the second period, A14 is the amplitude of the signal at the starting time of the third period, the starting time Φ of the second predetermined interval Δt2 is 0°, and the termination time Φ of the second predetermined interval Δt2 is 90°. In FIG. 7, the second signal S2 for the second period is a signal with a constant amplitude, so A13=A12. If the second signal S2 for the second period is an signal with a fluctuating amplitude as shown in FIG. 9, A13 and A12 may be different. In this solution, the amplitude change of the driving signal from the first signal S1 to the second signal S2 follows a sinusoidal curve, and similarly, the amplitude change of the signal from the second signal S2 to the third signal S3 follows a sinusoidal curve, so that the amplitude change of the driving signal can better adapt to the wall thickness change process in the process of sealing vessel. In this solution, the driving signal in FIG. 7 is illustrated by taking a current signal as an example. It should be understood that, since current and voltage can be converted according to the Ohm's law, the voltage can be obtained through conversion by combining the impedance with the current. Therefore, the driving signal provided in one of embodiments of the present application may also be configured as current signal or a voltage signal.

As shown in FIG. 7, in the case that the ultrasonic surgical system is adapted for sealing the vessel, the changes of amplitude of the signals in three stages are as follows:
the end effector assembly 40 is driven to perform the stage of separating muscle tissue layer of the vessel: the driving signal is provided with a constant amplitude to quickly dry the vessel wall. The duration of this stage may be determined according to the time when the muscular layer separation of the vessel is completed, and whether the muscular layer separation of the vessel is completed or not may be determined according to the impedance fed back by the end effector assembly 40. In this stage, the ultrasonic energy with constant power provided by the blade 41, and combined with the force exerted by the operator, can separate the muscle layer of the vessel without causing significant heat damage.

The end effector assembly 40 is driven to perform the stage of sealing and coagulating the vessel: the amplitude of the driving signal may be provided with a constant amplitude to coagulate the vessel. The duration of this stage may be determined according to the time when the coagulation of the vessel is completed, and whether the coagulation of the vessel is completed or not may be determined according to the impedance fed back by the end effector assembly 40.

The end effector assembly 40 is driven to perform the stage of cutting the vessel: the driving signal is provided with a constant amplitude (the constant amplitude may be the same as or different from the constant amplitude used in the stage of separating muscle layer of the vessel) until the vessel is transected.

In the above three stages, the duration of signals in different stages may be determined according to the impedance changes fed back by the end effector assembly 40 during the process of sealing vessel. Therefore, as shown in FIG. 8, the method for driving the ultrasonic surgical instrument may further comprise the following steps:

At S40, variations of a current value or voltage value fed back by the ultrasonic surgical instrument is acquired, wherein the current value or voltage variation is determined according to an impedance variation fed back by the end effector assembly 40. With reference to FIG. 3 and the principle of ultrasonic energy generation and transmission in the ultrasonic surgical system, during the process of sealing vessel, the property change of the vessel wall (such as the wall thickness of the vessel, tissue characteristics of the vessel, etc.) leads to the impedance change of the end effector assembly 40, which can be fed back to the transducer 10 along a transmission path of the ultrasonic vibration to cause the current value or voltage value of the transducer 10 to change. This step is performed in real time during the execution of the process of sealing the vessel. In which, the acquired current value or voltage variation is provided by the transducer 10.

At S50, the amplitude of the signal in each period is adjusted according to the current value or voltage variation, wherein each period comprises the first period, the second period, the third period, the first predetermined interval and/or the second predetermined interval. In this step, the adjusted period is determined according to the current operation stage performed by the end effector assembly 40, for example, when the end effector assembly 40 performs the stage of separating the muscle tissue layer of the vessel, the amplitude of the signal output in the first period is adjusted according to the current value or voltage variation; when the end effector assembly 40 performs the stage of sealing and coagulating the vessel, the amplitude of the signal output in the second period is adjusted according to the current value or voltage variation.

Further, the above method may also comprise the following step: determining time lengths of the first period, the second period and the third period according to the current or voltage variation. As in step S50, in this step, the adjusted period is determined according to the operation stage currently performed by the end effector assembly 40, that is, the amplitude of the signal is adjusted in real time according to the current or voltage variation fed back by the ultrasonic surgical instrument, or the waveform of the signal is adjusted in real time in the process of sealing the vessel.

Alternatively, in the above method, step S60 of adjusting the duration of the transition signal is further included in the stage of adjusting the end effector assembly 40 to perform the stage of sealing and coagulating the vessel, specifically: in the case that the first predetermined interval Δt1 is above zero (i.e. greater than zero), the above method comprises S601: determining the time length of the first predetermined interval Δt1 according to the current variation or voltage variation.

If the second predetermined interval Δt2 is above zero (i.e. greater than zero), the above method comprises S602: determining the time length of the second predetermined interval Δt2 according to the current variation or voltage variation.

In addition, in the process of realization, the first predetermined interval Δt1 and the second predetermined interval Δt2 may be equal or different. If the first interval equals to the second interval, the waveform may be simplified. If the first interval is different from the second interval, a difference between the two intervals is within a certain allowable error range, and the allowable error range may be determined according to empirical values or experimental calibration.

As described above, the impedance value of the end effector assembly 40 is also changing due to the change of the vessel tissue characteristics in the stage of sealing and coagulating vessel by the ultrasonic surgical system. Therefore, the second signal S2 is designed as a signal with volatility, that is, the ultrasonic energy of the blade 41 has a small fluctuation, so that the end effector assembly 40 can better reflect the impedance change in the process of sealing vessel, and a small fluctuation range can avoid unnecessary tearing of the vessel or tissue caused by the energy fluctuation of the blade. Alternatively, a difference between an upper limit A5 and a lower limit A6 of the second amplitude range of the second signal S2 is smaller than a set value. Taking the waveform diagram shown in FIG. 9 as an example, the signal provided over the second period is configured as a periodic signal, specifically, the periodic signal may be represented by the following function: B=A5−set value×sin(π×τ/T); wherein B is the amplitude of the signal output in the second period, τ is a time variable, τ∈(0, T), T is a time length of the second period. In the above, because the end effector assembly 40 needs to continuously act on the vessel with low energy to completely coagulate the vessel in the stage of sealing and coagulating vessel, in this stage, the ultrasonic energy and duration of the blade 41 need to be well matched, that is, the amplitude and the duration of the signal in the second period need to be well matched, so as to avoid the carbonization of vessel caused by excessively high amplitude or long duration of the signal in the second period and influence the sealing and coagulation effect. On this premise, the selection of the set value in the above function is compatible with the lower energy value required by the blade 41 in the coagulation stage of the vessel, which can be obtained through calibration experiments or empirical values. In this step, the amplitude of the signal in the second signal S2 is designed according to an impedance disturbance law in the process of sealing vessel, and the periodic fluctuation in sine wave mode can better reflect the impedance change in the process of sealing and coagulating vessel.

The embodiments of the present application further provide a generator for driving an ultrasonic surgical instrument. FIG. 10a to 10b are schematic diagrams of the generator 50 and other members when the generator 50 is used in an ultrasonic surgical system. The generator 50 comprises a processor chip 501 (such as a single chip microcomputer, a PLC, a DSP, and the like), and the processor chip 501 is pre-loaded with program information for generating various preset waveform signals. When the generator 50 responds to an initiating signal, the processor chip 501 executes the program information to perform the method steps provided in the above method embodiments, so that the generator 50 outputs a driving signal as shown in FIG. 5a to FIG. 5e, or FIG. 7 and FIG. 9 to the ultrasonic surgical instrument. The processor chip 501 can directly output waveform signals that meet the requirements of the driving signal through the preset program information, that is, the amplitude and duration of each stage of the signal meet the driving requirements of the ultrasonic surgical instrument. Alternatively, as shown in FIG. 10b, the processor chip 501 is connected with a signal conditioning circuit 502, and the processor chip 501 outputs a preset waveform signal (as an initial signal) after executing the program information, and the signal conditioning circuit 502 processes the preset waveform signal into the driving signal. In this solution, the amplitude of the preset waveform signal may be small, and the signal conditioning circuit 502 may have a signal amplification function, which can amplify an initial signal with a smaller amplitude into a driving signal meeting the driving requirements. A function of the signal conditioning circuit 502 is adapted to the initial signal output by the processor chip 501 and the driving requirements, and besides the amplification function, the signal conditioning circuit may also have transformation and filtering functions.

Further, the generator 50 further comprises an impedance detection circuit 503. The impedance detection circuit 503 is connected with the transducer 10 of the ultrasonic surgical instrument. The impedance detection circuit 503 is used for detecting a current variation or voltage variation fed back by the transducer 10, converting the current variation or voltage variation into a digital signal and feeding the digital signal back to the processor chip 501, wherein the current variation or voltage variation is determined according to the impedance variation of the end effector assembly 40. The processor chip 501 adjusts an amplitude of the driving signal according to the digital signal fed back by the impedance detection circuit 503. Further, the processor chip 501 can also adjust the time lengths of a first period, a second period and a third period in the driving signal according to the digital signal fed back by the impedance detection circuit 503, and determine the time length of the first predetermined interval according to the current variation or voltage variation when the first predetermined interval is above zero or greater than zero. And/or, the time length of the second period is determined according to the current variation or voltage variation when the second predetermined interval is above zero or greater than zero.

The above generator for driving the ultrasonic surgical instrument can output the driving signal to the end effector assembly 40 of the ultrasonic surgical instrument. The blade 41 in the end effector assembly 40 is driven by the driving signal to generate ultrasonic energy suitable for different stages in the process of sealing vessel, and a clamping force exerted by an operator on the end effector assembly 40 can be combined to quickly complete vessel sealing.

The embodiments of the present application further provide an ultrasonic surgical system. The system comprises an ultrasonic surgical instrument and the generator 50 provided by the embodiments above. The ultrasonic surgical instrument comprises a transducer 10 and an operating assembly. The transducer 10 receives the driving signal output by the generator 50 and converts the driving signal into an ultrasonic vibration signal. The operating assembly is provided with a waveguide 31 and an end effector assembly 40 arranged at a distal end of the waveguide 31 and used for operating a tissue. The ultrasonic vibration signal is transmitted from the waveguide 31 to the end effector assembly 40, and the ultrasonic energy generated by the end effector assembly 40 acts on the operated tissue. In the embodiments of the present application, a vessel is taken as the operated tissue as an example, but the ultrasonic surgical system can be applied to the cutting, coagulation and/or clamping of other tissues except vessel.

As shown in FIG. 10b, the ultrasonic surgical system may further comprise an initiating signal switch 50A (such as an operating hand switch 24, a foot switch connected to the generator 50, and the like). When the generator 50 receives the initiating signal from the initiating signal switch 50A (a signal receiving end of the processor chip 501 is used as a signal receiving end of the generator 50 in the figure), the processor chip 501 can execute the preset program information pre-stored therein and output a preset waveform signal. The preset waveform signal is processed by the signal conditioning circuit 502 to form a driving signal and input into the transducer 10. After ultrasonic vibration, the blade 41 in the end effector assembly 40 cooperates with a jaw assembly 42 to clamp the tissue, and the impedance change of the end effector assembly 40 is fed back to the transducer 10 to cause a current variation or voltage variation of the transducer 10. The impedance detection circuit 503 can detect the current variation or voltage variation, process the current variation or voltage variation into a digital signal and feed the digital signal back to the processor chip 501. The processor chip 501 can determine an impedance variation according to the current value or voltage variation, adjust an initial waveform according to the impedance variation, and change the amplitude of the driving signal to change the ultrasonic energy of the end effector assembly 40, so that the end effector assembly 40 can generate ultrasonic energy corresponding to the characteristic variation of the operated tissue at any stage, and better meet the operating requirements of the operated tissue.

In addition, the driving current waveforms proposed in the above embodiments of the present application are expressed according to an effective value of current, and the effective value is a value used to measure a magnitude of an alternating current. The specific calculation process is as follows: when the alternating current passes through a resistor, heat generated in one period is equal to heat generated by a direct current passing through the resistor in the same time, and a magnitude of the direct current is the effective value of the alternating current, so the effective value can be calculated according to an instantaneous value of the current. In specific applications, the instantaneous value or the effective value may be selected as a variable for display. It is verified that when the driving signal in the above embodiments of the present application is used to control the blade 41 to work, in the process of sealing and coagulating a larger diameter vessel, when the signals in each stage work at resonance frequency, and both ends of the second signal S2 for the second period adopt sine wave curves to realize the transition of the signals, the ultrasonic vibration energy of the blade 41 can achieve a maximum output power value in a unit time on the premise of meeting the needs of vessel sealing, so that the whole sealing process takes a short time.

Based on the driving signal in the above embodiments, a vibration duration of the blade 41 can be effectively shortened, and the heat generated by the mechanical vibration of the blade 41 can be reduced. Compared with the structure of the existing blade 41 with a heat dissipation film (because the blade needs to vibrate for a longer time in the prior art, more heat will be generated, so the blade needs to be additionally provided with the heat dissipation film), the present application can realize that: the exterior of the blade 41 does not need to be coated with a heat dissipation film. The heat dissipation film coated on the existing blade 41 not only increases the instrument cost, but also may fall off into a human body under the condition of high-frequency vibration, which is easy to cause rejection of the human body. In this solution, the blade 41 effectively solves the above problems after omitting the heat dissipation film.

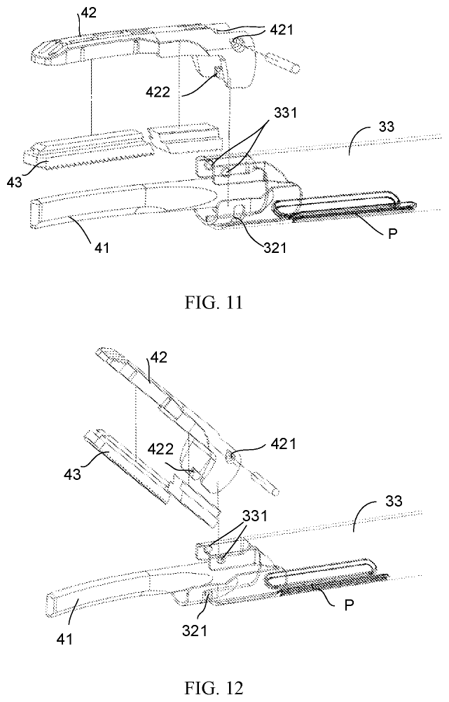

In the above-mentioned ultrasonic surgical system, when the end effector assembly 40 clamps the vessel, the blade 41 needs to be matched with the jaw assembly 42 to apply a loading force to the tissue in a conventional use scenario (such as the sealing and coagulation of a vessel with a diameter of about 5 mm or less). Specifically, as shown in FIG. 11 and FIG. 12, the pivotal movement of the jaw assembly 42 relative to the blade 41 is realized by setting a pair of pivot points on the jaw assembly 42, which are respectively engaged with the outer tube 33 and the inner tube 32. The outer tube 33 is fixedly connected to the handle assembly 20. The jaw assembly 42 is pivotally connected to the outer tube 33 via a first through hole 421 on the jaw assembly 42 and a corresponding second through hole 331 on the outer tube 33. A fastening pin or rivet slides through the first through hole 421 and the second through hole 331 to pivotally connect the jaw assembly 42 to the outer tube 33. The inner tube 32 moves along a longitudinal axis of the outer tube 33. A pivot pin 422 on the jaw assembly 42 engages a pivot hole 321 at a distal end of the inner tube 32. Therefore, the reciprocating movement of the inner tube 32 relative to the outer tube 33 makes the jaw assembly 42 pivot relative to the blade 41. The movement of the trigger 23 toward the handgrip 22 moves the inner tube 32 proximally, thereby pivoting the jaw assembly 42 toward the blade 41. A pulling action provided by the trigger 23 and the cooperative handgrip 22 helps conveniently and effectively manipulate and position the instrument, and operate the jaw assembly 42 at the distal end of the instrument to pivot toward the blade 41 side, whereby the tissue is effectively driven and pushed against the blade 41. The movement of the trigger 23 away from the handgrip 22 moves the inner tube 32 distally, thereby pivoting the jaw assembly 42 away from the blade 41.

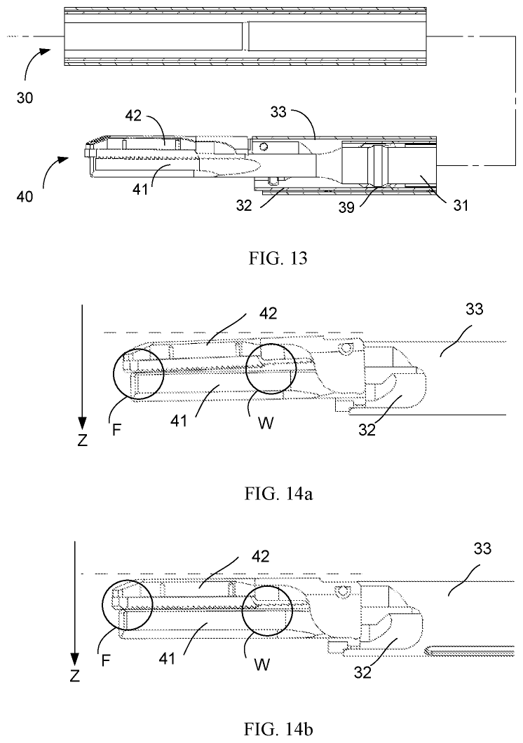

As shown in FIG. 13, a plurality of grooves or notches are formed in an outer periphery of the waveguide 31 for mounting sealing supporting portions 39. The grooves are located at a node of the waveguide 31. Since the ultrasonic amplitude at the node of the waveguide 31 is zero, the sealing supporting portions 39 are provided at this position, which can effectively support the waveguide 31 without affecting the ultrasonic transmission of the waveguide 31. The sealing supporting portion 39 is specifically a sealing rubber ring arranged in the groove, and the sealing rubber ring is made of flexible materials such as silica gel. The sealing supporting portion 39 provided at a farthest node is closest to the end effector assembly 40, and the sealing supporting portion 39 can also prevent a tissue residue generated during the end effector assembly 40 cutting from entering into the transmission assembly 30 through an area between the waveguide 31 and the inner tube 32.

When the end effector assembly 40 is used for sealing and coagulating vessel with a larger diameter, it is necessary to operate the trigger 23 with a greater force to clamp the vessel. The force of the jaw assembly 42 in a Z direction is greater, so that the blade 41 tends to move in the Z direction. A assembly gap between the inner tube and the outer tube allows the blade 41 to move in the Z direction. As shown in FIG. 13, the waveguide 31, which is fastened or integrated with the blade 41, abuts against the inner tube 32 through the sealing supporting portion 39 without clearance, and an assembly gap is provided between the inner tube and the outer tube. As shown in FIG. 14a, a straight line formed by the above pair of pivot points inclines to the left (or it may also be considered that the straight line inclines to the right). At this time, the distal end F of the end effector assembly 40 is closed, but there is still a large gap at the proximal end W of the end effector assembly. There is a big gap at the proximal end W, and if the end effector assembly clamps the vessel, the pressure is low. At this time, it is easy to see that the vessel clamped at the distal end has finished transection/hemostasis under the action of pressure and energy, while the vessel clamped at the proximal end has not finished transection/hemostasis. That is, the force consistency of the end effector assembly 40 acting on the vessel with larger diameter is poor, and the problem that some vessels are not cut off or coagulated easily occurs.

In the above embodiments provided by the present application, the driving signal can control the blade 41 to output high enough ultrasonic energy per unit time, so that the operator can appropriately reduce the clamping force from the operator when controlling the action of the jaw assembly 42, thus alleviating poor consistency of the loading force of the end effector assembly 40.

In order to further make the loading situation of the blade 41 from the proximal end to the distal end consistent when the end effector assembly 40 seals and coagulates the vessel with larger diameter, it is necessary to make the force applied on the blade 41 gradually decrease from the proximal end to the distal end. Alternatively, in this embodiment, a butting part is provided at the distal ends of the inner tube 32 and the outer tube 33, and the butting part forms a support between the inner tube 32 and the outer tube 33, so that a gap between the distal ends of the inner tube and the outer tube is close to zero, which can prevent radial gap between the inner tube and the outer tube from changing without affecting the relative sliding movement of the inner tube and the outer tube, that is, the displacement of the inner tube 32 along the Z direction is close to zero when the blade 41 is subjected to the force exerted by the jaw assembly 42 along the Z direction, which can prevent radial gap between the inner tube and the outer tube from changing. As shown in FIG. 14b, when the jaw assembly 42 pivots in a direction close to the blade 41 to seal and coagulate the larger diameter vessel, position displacement of the blade 41 in the radial Z direction decreases with the support of the distal ends of the inner tube 32 and the outer tube 33, which is almost zero. At this time, the distal end F of the end effector assembly 40 is closed, and the gap at the proximal end W of the end effector assembly is small, so that the force on the blade 41 is gradually increased from the distal end to the proximal end. More specifically, an inner side of the butting part abuts against an outer wall of the inner tube 32 or is integrally formed with the outer wall of the inner tube 32, and an outer side of the butting part is integrally formed with an inner wall of the outer tube 33 or abuts against the inner wall of the outer tube 33. In this way, the gap between the inner tube 32 and the outer tube 33 is further reduced by providing the butting part on the distal end side. In some schemes, the butting part is at least one clamping piece P arranged between the inner tube 32 and the outer tube 33. By arranging the clamping piece P independent of the inner tube 32 and the outer tube 33, processing difficulty of the inner tube 33 and the outer tube 33 can be reduced. More specifically, a surface of the clamping piece P is shaped as an arc-shaped surface matched with the walls of the inner tube and the outer tube 33, so that the clamping piece P has a large contact area with the outer wall of the inner tube 32 and the inner wall of the outer tube 33 to stably support the waveguide 31. It may be understood that the implementation of the above butting part is only a schematic illustration, and the clamping piece P may be replaced with other structural members that can achieve the same function in the concrete implementation.

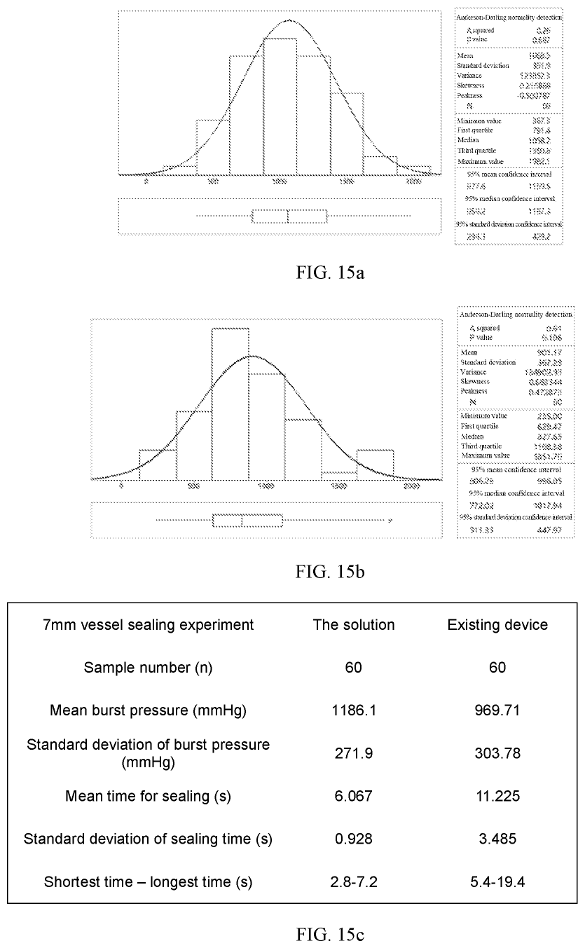

According to the above solution in this embodiment, by improving the mechanical structure of the end effector assembly 40 and in combination with the improved driving signal, the time required to seal the larger diameter vessel is further shortened, and the consistency of the loading force of the end effector assembly 40 is improved. In the process of realizing this solution, the ultrasonic surgical instrument provided in this embodiment and the ultrasonic surgical instrument in the prior art are selected to perform sealing and coagulating experiment on 60 vessel samples with a diameter of about 7 mm, and the effect of this solution is explained from two aspects comprising time required for sealing operation and a verification result of a burst pressure of the sealed vessel. Specifically, the two instruments are used to perform the sealing and coagulating on a plurality of vessel samples with a diameter of about 7 mm. After the whole sealing and coagulating process is completed, the burst pressure is measured at the position where the vessel is sealed, and different time required for different sealing operations corresponding to different burst pressure experimental results is counted respectively. Finally, the verification results of this solution are shown in FIG. 15a, the verification results of the existing product are shown in FIG. 15b, and the comparison results are shown in FIG. 15c, wherein:

The time of performing the sealing operation of the ultrasonic surgical instrument provided in this solution is within a range of 2.8-7.2s, a mean time of performing the sealing operation is 6.067s and a standard deviation of the time of performing the sealing operation is 0.928s. After completing the vessel sealing operation, a mean burst pressure at the position where the vessel is sealed is 1186.1 mmHg, and the standard deviation obtained according to the burst pressure experimental results is 271.9 mmHg.

The time of performing the sealing operation of the existing products is within a range of 5.4-19.4s, a mean time of performing the sealing operation is 11.225s and a standard deviation of the time of performing the sealing operation is 3.485s. After completing the vessel sealing operation, a mean burst pressure at the position where the vessel is sealed is 969.71 mmHg, and the standard deviation of the burst pressure is 303.78 mmHg.

According to the experimental results shown in the figure, when sealing the vessel with a diameter of about 7 mm, the solution of the present application can obviously use a shorter sealing time and get a better sealing result. With the solution, the vessel sealing efficiency can be further improved on the premise of ensuring the quality of the vessel sealing.

What is claimed is:

1. A method for driving an ultrasonic surgical instrument, wherein the method comprises:
in response to an initiating signal, providing a drive signal with a first amplitude range for a first period to drive an transducer of the ultrasonic surgical instrument;
subsequent to the first period, providing a first transition signal with a decreasing amplitude for a first predetermined interval to drive the transducer;
subsequent to the first predetermined interval, providing a drive signal with a second amplitude range for a second period to drive the transducer, wherein an upper limit of the second amplitude range is lower than a lower limit of the first amplitude range;
subsequent to the second period, providing a second transition signal with an increasing amplitude for a second predetermined interval to drive the transducer; and
subsequent to the second predetermined interval, providing a drive signal with a third amplitude range for a third period to drive the transducer, wherein a lower limit of the third amplitude range is higher than the upper limit of the second amplitude range;
wherein at least one of the first predetermined interval and the second predetermined interval is above zero.

2. The method for driving the ultrasonic surgical instrument according to claim 1, wherein if the first predetermined interval is above zero, the amplitude of the first transition signal provided to the ultrasonic surgical instrument for the first predetermined interval decreases following a first nonlinear curve.

3. The method for driving the ultrasonic surgical instrument according to claim 2, wherein the first nonlinear curve is defined as A=(A11−A12)×sin Φ+A11, Φ∈(180°, 270°), wherein A is the amplitude of the first transition signal, A11 is the amplitude of the signal at the termination of the first period, and A12 is the amplitude of the signal at the starting of the second period.

4. The method for driving the ultrasonic surgical instrument according to claim 1, wherein if the second predetermined interval is above zero, the amplitude of the second transition signal provided to the ultrasonic surgical instrument for the second predetermined interval increases following a second nonlinear curve.

5. The method for driving the ultrasonic surgical instrument according to claim 4, wherein the second nonlinear curve is defined as C=(A14−A13)×sin Φ+A13, Φ∈(0°, 90°), wherein C is the amplitude of the second transition signal, A13 is the amplitude of the signal at the termination of the second period, and A14 is the amplitude of the signal at the starting of the third period.

6. The method for driving the ultrasonic surgical instrument according to claim 1, wherein if the first predetermined interval is above zero, the amplitude of the first transition signal provided to the ultrasonic surgical instrument for the first predetermined interval decreases linearly.

7. The method for driving the ultrasonic surgical instrument according to claim 1, wherein if the second predetermined interval is above zero, the amplitude of the second transition signal output to the ultrasonic surgical instrument in the second predetermined interval increases linearly.

8. The method for driving the ultrasonic surgical instrument according to claim 1, wherein a difference between an upper limit and a lower limit of the second amplitude range of the signal provided to the ultrasonic surgical instrument for the second period is lower than a set value.

9. The method for driving the ultrasonic surgical instrument according to claim 8, wherein the signal provided to the ultrasonic surgical instrument for the second period is a periodic signal.

10. The method for driving the ultrasonic surgical instrument according to claim 9, wherein the periodic signal provided to the ultrasonic surgical instrument for the second period is a sine wave signal.

11. The method for driving the ultrasonic surgical instrument according to claim 10, wherein the sine wave signal provided to the ultrasonic surgical instrument for the second period is represented by the following function: B=A5−set value×sin(π×τ/T); wherein B is the amplitude of the signal for the second period, τ is a time variable, τ∈(0, T), T is a time length of the second period, and A5 is an upper limit of the amplitude of the signal for the second period.

12. The method for driving the ultrasonic surgical instrument according to claim 1, wherein the method further comprises:
acquiring a current variation or voltage variation fed back by the ultrasonic surgical instrument, wherein the current variation or voltage variation is determined according to an impedance variation of the end effector assembly; and
adjusting the amplitude of the signal in each period according to the current variation or voltage variation, wherein each period comprises the first period, the second period, the third period, the first predetermined interval and/or the second predetermined interval.

13. The method for driving the ultrasonic surgical instrument according to claim 12, wherein the method further comprises determining time lengths of the first period, the second period and the third period according to the current variation or voltage variation.

14. The method for driving the ultrasonic surgical instrument according to claim 13, wherein the method further comprises:
determining the time length of the first predetermined interval according to the current variation or voltage variation if the first predetermined interval is above zero; and/or,
determining the time length of the second predetermined interval according to the current variation or voltage variation if the second predetermined interval is above zero.

15. The method for driving the ultrasonic surgical instrument according to claim 1, wherein at least one of the signals over the first period, the second period and the third period has a frequency that is a resonance frequency of the ultrasonic surgical instrument.

16. A generator for driving an ultrasonic surgical instrument, wherein the generator comprises a processor chip, the processor chip is pre-loaded with program information, and when the generator responds to an initiating signal, the processor chip executes the program information to perform the method for driving the ultrasonic surgical instrument according to claim 1, so that the generator outputs a driving signal to the ultrasonic surgical instrument.

17. The generator for driving the ultrasonic surgical instrument according to claim 16, wherein the generator further comprises a signal conditioning circuit, the processor chip outputs a preset waveform signal after executing the program information, and the signal conditioning circuit processes the preset waveform signal into the driving signal.

18. The generator for driving the ultrasonic surgical instrument according to claim 16, wherein the generator further comprises:
an impedance detection circuit, the impedance detection circuit is used for detecting a current variation or voltage variation fed back by the ultrasonic surgical instrument, converting the current variation or voltage variation into a digital signal and feeding the digital signal back to the processor chip, wherein the current variation or voltage variation is determined according to the impedance variation of the end effector assembly; and
the processor chip adjusts an amplitude of the driving signal according to the digital signal fed back by the impedance detection circuit.

19. The generator for driving the ultrasonic surgical instrument according to claim 18, wherein the processor chip adjusts time lengths of a first period, a second period and a third period of the driving signal according to the digital signal fed back by the impedance detection circuit.

20. The generator for driving the ultrasonic surgical instrument according to claim 19, wherein the processor chip determines the time length of the first predetermined interval according to the current variation or voltage variation when the first predetermined interval is above zero; and/or, determines the time length of the second predetermined interval according to the current variation or voltage variation when the second predetermined interval is above zero.
